SEQUENCE LISTING (1 sequence)

SEQ ID NO: 1 - DNA, 939 bp, Homo sapiens
atgaagacgatcatcgccctgagctacatcttctgcctggtattcgccgactacaaggacgatgatgacaaggggatcttagacctagccccacagatgcttcgagaactccaggagactaatgcggcgctgcaagacgtgagagagctcttgcgacagcaggtcaaggagatcaccttcctgaagaatacggtgatggaatgtgacgcttgcggaggatcccttgtcaatctttgcactaaagaaggtgttttactaaagggaggaaaaagagaggaagagaaaccatttagagactgtgcagatgtatatcaagctggttttaataaaagtggaatctacactatttatattaataatatgccagaacccaaaaaggtgttttgcaatatggatgtcaatgggggaggttggactgtaatacaacatcgtgaagatggaagtctagatttccaaagaggctggaaggaatataaaatgggttttggaaatccctccggtgaatattggctggggaatgagtttatttttgccattaccagtcagaggcagtacatgctaagaattgagttaatggactgggaagggaaccgagcctattcacagtatgacagattccacataggaaatgaaaagcaaaactataggttgtatttaaaaggtcacactgggacagcaggaaaacagagcagcctgatcttacacggtgctgatttcagcactaaagatgctgataatgacaactgtatgtgcaaatgtgccctcatgttaacaggaggatggtggtttgatgcttgtggcccctccaatctaaatggaatgttctatactgcgggacaaaaccatggaaaactgaatgggataaagtggcactacttcaaagggcccagttactccttacgttccacaactatgatgattcgacctttagatttttga

(19) United States
(12) Patent Application Publication
(10) Pub. No.: US 2006/0024825 A1
(43) Pub. Date: Feb. 2, 2006

(54) METHOD OF PROMOTING EXPANSION OF STEM CELLS

(75) Inventors: Yoon-Sun Yang, Seoul (KR); Wonil Oh, Seoul (KR); Sung-Eun Yang, Seoul (KR)

Correspondence Address: JHK LAW, P.O. BOX 1078, LA CANADA, CA, 91012-1078, US

(73) Assignee: MEDIPOST CO. LTD., Seoul (KR)

(21) Appl. No.: 11/161,195

(22) Filed: Jul. 26, 2005

Related U.S. Application Data
(60) Provisional application No. 60/591,303, filed on Jul. 26, 2004.

(51) Int. Cl. A61K 38/18; C12N 5/08

(52) U.S. Cl. 435/372; 514/12

(57) ABSTRACT

The present application discloses a method for expanding a population of mammalian cells comprising contacting the cells with angiopoietin 1.

### CROSS-REFERENCE To RELATED APPLICATIONS

The present application claims priority to U.S. Provisional Application No. 60/591,303, filed Jul. 26, 2004, the contents of which are incorporated by reference herein in their entirety.

### BACKGROUND OF THE INVENTION

1. Field of the Invention

The present invention relates to an ex vivo method for expanding mammalian stem cell population. The invention also relates to using angiopoietin 1 or a coiled coil domain linked to a fragment of angiopoietin 1 as stem cell population expansion effective compound.

2. General Background and State of the Art

Hematopoietic stem cells (HSCs) are generally defined as cells having the self-renewing potential and the capacity to give rise to differentiated cells of all hematopoietic lineages (Osawa M et al., Science. 1996;273:242-245). Therefore, HSC transplantation is performed for complete healing of hematologic disorders and as a supportive therapy after high-dose chemotherapy against malignant diseases. HSCs can be collected from peripheral blood (PB), bone marrow (BM), and cord blood (CB). Although human CB is thought to contain a high number of primitive hematopoietic cells, the total number of CB HSCs harvested from one donor's umbilical CB is limited and is not sufficient for HSC transplantation in an adult patient. To overcome this problem, attention has been increasingly focused on ex vivo expansion of HSCs.

Many approaches have been reported during the last decade, and they can be generally divided into two categories. The first category is treatment of HSCs with various combinations of cytokines. Treatment with the following combinations of cytokines increased the progenitor/stem cell population by 2- to 30-fold in the relatively short period of 10 to 14 days: Flk-2/Flt-3 ligand (FL), stem cell factor (SCF), and thrombopoietin (TPO); SCF, granulocyte-colony stimulating factor (G-CSF), and megakaryocyte growth and development factor (MGDF); FL, SCF, G-CSF, interleukin-3 (IL-3), and IL-6; and FL, SCF, and IL-6 (Petzer A L et al., J Exp Med. 1996; 183:2551-2558; McNiece I et al., Exp Hematol. 2000; 28:1181-1186; Conneally E et al., Proc Natl Acad Sci U S A. 1997; 94:9836-9841; Ueda T et al., J Clin Invest. 2000; 105:1013-1021). However, it is difficult to maintain HSC activity in long-term cultures even if the total number of hematopoietic cells could be expanded. Hence, these methods could be improved for use in clinical settings.

The second category involves using stromal cells. Several methods of ex vivo expansion using human primary stromal cells were reported (Gan O I et al., Blood. 1997; 90:641-650; Yamaguchi M et al., Exp Hematol. 2001; 29:174-182). When HSCs were cocultured with human primary stromal cells, the HSCs were expanded for 2 to 4 weeks. However, HSCs have frequently lost their stemness during ex vivo expansion. Maintenance of stemness, number, proliferative activity of HSCs are critical for transplantation. CD34+ cells are generally known to be HSCs or hematopoietic precursor cells.

It is known that the Tie2 receptor is expressed not only by endothelial cells but also by HSCs, indicating another possible role of angiopoietin-1 (Ang1) and Tie2 in hematopoiesis (Iwama et al., Biochem Biophys Res Commun 195:301-309, 1993). Tie2 deficient mice show severely impaired hematopoiesis (Sato et al., Nature 376:70-74, 1995). In addition, Takakura et al. discovered that soluble Tie2 receptor inhibited hematopoiesis in para-aortic splanchnopleural mesoderm explant culture (Takakura et al., Immunity, 9:677-686, 1998). In addition, Ang1 induced adhesion of Tie2-expressing HSCs to fibronectin leads to hematopoietic proliferation (Takakura et al., Immunity, 9:677-686, 1998). Moreover, Tie2 on HSCs also may be critical in the interaction of these cells with endothelial cells (Takakura et al., Cell 102:199-209, 2000; Phillips et al., Science 288:1635-1640, 2000). In fact, HSCs closely adhere to endothelial cells at several sites in the embryo. Furthermore, it has been found that HSCs produce Ang1, suggesting that HSCs can promote the migration of endothelial cells and establish the hematopoietic environment (Takakura et al., Cell 102:199-209). In addition, Ang1 promoted the adhesion of sorted primary Lin (−/low)CD34(+)TIE2(+) cells to fibronectin (FN), and this adhesion may play a critical role in keeping HSCs in an immature status under the stromal cells (Yuasa H et al., BBRC 298:731-737, 2002). However, large-scale production of recombinant Ang1 is hindered by the aggregation and insolubility of the protein (Procopio W N et al., J Biol Chem. 274, 30196-30201, 1999; Davis, S. et al. Nat. Struct. Biol. 10, 38-44, 2003). The activity of the protein frequently varies after purification. These difficulties are due to its unique structural characteristics. Recently, Cho et al. (PNAS 101; 5547-5552, 2004) have developed a soluble, stable and potent Ang1 variant, COMP-Ang1. The contents of Cho et al. are incorporated by reference herein in their entirety especially with respect to the production of the COMP-Ang1 chimera.

Therefore, there is a continuing need in the art to generate stem cells that are useful for medical treatment.

### SUMMARY OF THE INVENTION

The invention overcomes the above-mentioned problems, and provides a compound and method for increasing the number of stem cells in a sample.

In one aspect, the present invention is directed to using COMP-Ang1 for ex vivo expansion of hematopoietic stem cells in the mesenchymal stromal cell culture system originated from human umbilical cord blood. In another aspect, the data show that COMP-Ang1 (800 ng/ml) promotes an increase in number of CD34+ cells by approximately 1.94 fold compared to control medium-treated group. In one aspect, the present invention is directed to a method for expanding a population of mammalian cells comprising contacting the cells with angiopoietin 1. The angiopoietin 1 may be a chimeric angiopoietin 1, and the angiopoietin may be substituted at the N-terminus with a coiled-coil domain. Further, the coiled-coil domain may be from cartilage oligomeric matrix protein (COMP).

In one aspect of the invention, the expansion may be in vivo, in vitro or ex vivo. And the mammalian cell may be a stem cell. The stem cell may be a totipotent or pluripotent stem cell. The totipotent stem cell may be an embryonic stem cell. The pluripotent stem cell may be hematopoietic stem cell (HSC) or mesenchymal stem cell. Further, the HSC may be without limitation CD34+ or CD45+.

In another aspect of the invention, the mammalian cell may be obtained from umbilical cord blood. And the cell may be the total nuclear cell of umbilical cord blood. Further, the mammal may be human.

In even another aspect of the invention, the angiopoietin 1 may be delivered to the cell via genetic vector format wherein the polypeptide molecule is expressed in a host cell and is secreted into the population.

The invention is further directed to a container which contains (a) a compound of coiled coil domain linked to Ang 1; and (b) instructions that cells be expanded by contacting the cells with the compound.

These and other objects of the invention will be more fully understood from the following description of the invention, the referenced drawings attached hereto and the claims appended hereto.

### DETAILED DESCRIPTION OF THE PREFERRED EMBODIMENTS

In the present application, “a” and “an” are used to refer to both single and a plurality of objects.

As used herein, administration “in combination with” one or more further stem cell proliferation agents includes simultaneous (concurrent) and consecutive administration in any order.

As used herein, “effective amount” is an amount sufficient to effect beneficial or desired clinical or biochemical results. An effective amount can be administered one or more times.

As used herein, “mammal” refers to any animal classified as a mammal, including humans, domestic and farm animals, and zoo, sports, or pet animals, such as dogs, cats, cattle, horses, sheep, pigs, and so on. Preferably, the mammal is human.

As used herein “pharmaceutically acceptable carrier and/or diluent” includes any and all solvents, dispersion media, coatings antibacterial and antifungal agents, isotonic and absorption delaying agents and the like. The use of such media and agents for pharmaceutical active substances is well known in the art. Except insofar as any conventional media or agent is incompatible with the active ingredient, use thereof in the stem cell proliferation effective compositions is contemplated. Supplementary active ingredients can also be incorporated into the compositions.

Coiled Coil

The α-helical coiled coil is probably the most widespread subunit oligomerization motif found in proteins. Accordingly, coiled coils fulfill a variety of different functions. In several families of transcriptional activators, for example, short leucine zippers play an important role in positioning the DNA-binding regions on the DNA (Ellenberger et al., 1992, Cell 71:1223-1237). Coiled coils are also used to form oligomers of intermediate filament proteins. Coiled-coil proteins furthermore appear to play an important role in both vesicle and viral membrane fusion (Skehel and Wiley, 1998, Cell 95:871-874). In both cases hydrophobic sequences, embedded in the membranes to be fused, are located at the same end of the rod-shaped complex composed of a bundle of long α-helices. This molecular arrangement is believed to cause close membrane apposition as the complexes are assembled for membrane fusion.

The coiled coil is often used to control oligomerization. It is found in many types of proteins, including transcription factors such as, but not limited to GCN4, viral fusion peptides, SNARE complexes and certain tRNA synthetases, among others. Very long coiled coils are found in proteins such as tropomyosin, intermediate filaments and spindle-pole-body components.

Coiled coils involve a number of α-helices that are supercoiled around each other in a highly organized manner that associate in a parallel or an antiparallel orientation. Although dimers and trimers are the most common. The helices may be from the same or from different proteins.

The coiled-coil is formed by component helices coming together to bury their hydrophobic seams. As the hydrophobic seams twist around each helix, so the helices also twist to coil around each other, burying the hydrophobic seams and forming a supercoil. It is the characteristic interdigitation of side chains between neighbouring helices, known as knobs-into-holes packing, that defines the structure as a coiled coil. The helices do not have to run in the same direction for this type of interaction to occur, although parallel conformation is more common. Antiparallel conformation is very rare in trimers and unknown in pentamers, but more common in intramolecular dimers, where the two helices are often connected by a short loop.

In the extracellular space, the heterotrimeric coiled-coil protein laminin plays an important role in the formation of basement membranes. Other examples are the thrombospondins and cartilage oligomeric matrix protein (COMP) in which three (thrombospondins 1 and 2) or five (thrombospondins 3, 4 and COMP) chains are connected. The molecules have a flower bouquet-like appearance, and the reason for their oligomeric structure is probably the multivalent interaction of the C-terminal domains with cellular receptors.

Chimeric Cartilage Oligomeric Matrix Protein (COMP)-Ang1

A non-collagenous glycoprotein, COMP, was first identified in cartilage (Hedbom et al., 1992, J. Biol. Chem. 267:6132-6136). The protein is a 524 kDa homopentamer of five subunits which consists of an N-terminal heptad repeat region (cc) followed by four epidermal growth factor (EGF)-like domains (EF), seven calcium-binding domains (T3) and a C-terminal globular domain (TC). According to this domain organization, COMP belongs to the family of thrombospondins. Heptad repeats (abcdefg)ₙ with preferentially hydrophobic residues at positions a and d form—helical coiled-coil domains (Cohen and Parry, 1994, Science 263:488-489). Recently, the recombinant five-stranded coiled-coil domain of COMP (COMPcc) was crystallized and its structure was solved at 0.2 nm resolution (Malashkevich et al., 1996, Science 274:761-765).

When mention is made of the chimeric construct COMP-Ang1, it is understood that the Ang1 portion referred to is a fragment of the Ang1, preferably a Tie2 binding domain, preferably at the carboxy domain of Ang1. This fragment may be at the fibrinogen domain of Ang1. Further, the COMP (cartilage oligomeric matrix protein) portion of COMP-Ang1 refers to the coiled coil domain of COMP. Further in particular, The coiled-coil domain of COMP may be 45-amino acids and may form a parallel pentamer.

It is understood that other coiled coil domains may be linked with Ang1 to produce a functional compound that results in stem cell population expansion upon contact with the cells. In this regard, COMP coiled coil domain may be considered to be one example of the usefulness of such a coiled coil domain.

Instructions and Kits

The present invention is also directed to a written medium which instructs the user to expand cells, in particular, umbilical cord blood cells or stem cells derived therefrom by contacting the cells with Angiopoietin 1 or a functionally equivalent molecule thereof. Such instructions may be written on a container which may contain the Angiopoietin 1 or cord blood cells or any related reagent thereof. Such instructions may be in written form in any medium, including paper, fax, computer, e-mail, website and so on. Further, such instructions may be posted in hospitals, companies, including cord blood preservation companies or universities.

The present invention is not to be limited in scope by the specific embodiments described herein. Indeed, various modifications of the invention in addition to those described herein will become apparent to those skilled in the art from the foregoing description and accompanying figures. Such modifications are intended to fall within the scope of the appended claims. The following examples are offered by way of illustration of the present invention, and not by way of limitation.

### EXAMPLES

### Example 1

### Materials and Methods

### Example 1.1

### Construction of the COMP/CC-Ang1/FD

COMP/CC-Ang1/FD consists of a hemagglutinin signal sequence at its amino terminus to allow for secretion (bases 1-48 of SEQ ID NO:1), a FLAG tag sequence (bases 49-72 of SEQ ID NO:1), a short bridging sequence consisting of the amino acids Gly-Ile-Leu (bases 73-81 of SEQ ID NO:1), the coding sequence of COMP coiled-coil domain (bases 82-221 of SEQ ID NO:1), another bridging sequence consisting of amino acids Gly-Ser (bases 222-227 of SEQ ID NO:1), and the coding sequence for the linker region of Ang1 (bases 228-296 of SEQ ID NO:1) followed by fibrinogen domain of Ang1 (FD) (bases 297-939 of SEQ ID NO:1).

The sequence of SEQ ID NO:1 is set forth below.

| | |
|---|---|
| atgaagacga tcatcgccct gagctacatc ttctgcctgg tattcgccga ctacaaggac | 60 |
| gatgatgaca aggggatctt agacctagcc ccacagatgc ttcgagaact ccaggagact | 120 |
| aatgcggcgc tgcaagacgt gagagagctc ttgcgacagc aggtcaagga gatcaccttc | 180 |
| ctgaagaata cggtgatgga atgtgacgct tgcggaggat cccttgtcaa tctttgcact | 240 |
| aaagaaggtg ttttactaaa gggaggaaaa agagaggaag agaaaccatt tagagactgt | 300 |
| gcagatgtat atcaagctgg ttttaataaa agtggaatct acactattta tattaataat | 360 |
| atgccagaac ccaaaaaggt gttttgcaat atggatgtca atgggggagg ttggactgta | 420 |
| atacaacatc gtgaagatgg aagtctagat ttccaaagag gctggaagga atataaaatg | 480 |
| ggttttggaa atccctccgg tgaatattgg ctggggaatg agtttatttt tgccattacc | 540 |
| agtcagaggc agtacatgct aagaattgag ttaatggact gggaagggaa ccgagcctat | 600 |
| tcacagtatg acagattcca cataggaaat gaaaagcaaa actataggtt gtatttaaaa | 660 |
| ggtcacactg ggacagcagg aaaacagagc agcctgatct tacacggtgc tgatttcagc | 720 |
| actaaagatg ctgataatga caactgtatg tgcaaatgtg ccctcatgtt aacaggagga | 780 |
| tggtggtttg atgcttgtgg cccctccaat ctaaatggaa tgttctatac tgcgggacaa | 840 |
| aaccatggaa aactgaatgg gataaagtgg cactacttca aagggcccag ttactcctta | 900 |
| cgttccacaa ctatgatgat tcgaccttta gatttttga | 939 |

Cord blood was divided into 50 ml Falcone tubes and centrifuged at 3,000 rpm for 20 min. Buffy coat layers containing MNCs were transferred by Pasteur pipette to PBS containing 2% FBS. Fifteen ml of PBS containing MNCs were overlaid to 25 ml of Ficoll-1077 (1.077 g/ml, Sigma-Aldrich), centrifuged at 1,300 rpm for 35 min, washed twice with PBS containing 2% FBS, removed RBC with lysis buffer (0.14M NH₄Cl, pH7.2), washed again twice with PBS containing 2% FBS, and separated MNCs was pooled. Then, CD34+ cells were enriched from the pooled MNCs using direct CD34 progenitor cell isolation kit according to manufacturer's protocol (Miltenyi Biotec.) with AutoMACS (Magnetic Cell Sorting System, Miltenyi Biotec). The enriched CD34+ cells were confirmed by FACS analysis after staining with anti human CD34-PE antibody.

### Example 1.3

### Preparation of Feeder Layer Cells

Feeder layer cells were prepared from mesenchymal cells originated from human cord bloods as previously described (Cytotherapy, 2004 Vol 6, No 5, in press). Mesenchymal cells (1˜3×10⁴ cells/cm²) were plated into 35 mm dish, incubated with α-MEM containing 10% FBS, and grown up to 90% confluence. Then the cells were incubated with mitomycin (10 μg/ml) and washed with PBS and α-MEM containing 10% FBS.

### Example 1.4

### Incubation of Enriched CD34+ Cells and Treatment of COMP-Ang1

Enriched CD34+ cells (5˜10×10⁴) were plated into 35 mm dish (feeder layer cells) and maintained in IMDM (Iscove's Modified Dulbecco's Medium) containing 10% FBS, β-mercaptoethanol (10⁻⁴ M), stem cell factor (100 μg/ml), thrombopoietin (10 μg/ml), Flt-3/Flk-2 ligand (50 μg/ml), interleukin-6 (100 μg/ml). In order to examine the effect of COMP-Ang1, indicated amounts of COMP-Ang1 were added to the enriched CD34+ cells for indicated days. The medium was refreshed twice a day and the cells were sub-cultured every week with new feeder layer cells.

### Example 1.5

### Cell Analysis

Morphology, number, viability and cell surface phenotypes of CD34+ cells were analyzed at 4, 7 and 14 days after incubation of indicated amount of COMP-Ang1. Expressions of CD45, CD34, CD38, CD33, CD41a, CD3, and CD19 in CD34+ cell surface were analyzed using FACS analysis.

### Example 2

### Results

### Example 2.1

### Effect of COMP-Ang1 on Morphology and Proliferative Activity of Total Nucleated Cells and CD34+ Cells

Phase contrast microscopic analysis revealed that COMP-Ang1 induced proliferation in a dose-dependent manner at day 7 with any notable alterations of morphology (FIG. 1). Although numbers of total nucleated cells (TNC) were increased on feeder layer culture at 4 and 7 days, there were no significant differences between control buffer-treated group and different concentrations of COMP-Ang1-treated groups (Table 1). However, at day 14, numbers of TNC were increased by COMP-Ang1 treatment in a dose-dependent manner (Table 1). Notably, 800 ng/ml of COMP-Ang1 increased number of TNC approximately 1.73 fold compared to control buffer-treated group.

TABLE 1
Number of total nucleated cells (×10⁶ cells)

| | w/o | | | | |
|---|---|---|---|---|---|
| | feeder | 0 ng/ml | 200 ng/ml | 400 ng/ml | 300 ng/ml |
| 0d | 0.07 | 0.07 | 0.07 | 0.07 | 0.07 |
| 4d | 0.53 | 0.42 | 0.57 | 0.4 | 0.49 |
| 7d | 4.65 | 3.6 | 4.32 | 3.65 | 4.5 |
| 14d | 39.3 | 32.6 | 37.2 | 41.2 | 56.4 |

Alternatively, the fold increase of TNC from day 0 to day 14 by control buffer, 200, 400, and 800 ng/ml of COMP-Ang1 were 465.4, 531.4, 588.4, and 805.2, while it was 561.3 without feeder layer culture (Table 2).

TABLE 2
Fold increase of total nucleated cells

| | W w/o | | | | |
|---|---|---|---|---|---|
| | feeder | 0 ng/ml | 200 ng/ml | 400 ng/ml | 800 ng/ml |
| 0d | 1.0 | 1.0 | 1.0 | 1.0 | 1.0 |
| 4d | 7.6 | 6.0 | 8.1 | 5.7 | 7.0 |
| 7d | 66.4 | 51.4 | 61.8 | 52.1 | 64.3 |
| 14d | 561.3 | 465.4 | 531.4 | 588.4 | 805.2 |

Although the fold increases of CD34+ cells were increased on feeder layer culture at day 7, there were no significant differences between control buffer-treated group and different concentrations of COMP-Ang1-treated groups (Table 3). However, at day 14, the fold increases of CD34+ cells were increased by COMP-Ang1 treatment in a dose-dependent manner (Table 3). The fold increase of CD34+ cells from day 0 to day 14 by control buffer, 200, 400, and 800 ng/ml of COMP-Ang1 were 208.5, 229.5, 262.6, and 404.1, while it was 163.9 without feeder layer culture (Table 3). Thus, notably, 800 ng/ml of COMP-Ang1 increased the fold increase number of CD34+ cells approximately 1.94 fold compared to control buffer-treated group, while it increased the fold increase number of CD34+ cells approximately 2.45 fold compared to control buffer-treated group without feeder layer culture.

TABLE 3
Fold increase of CD34+ cells

| | W w/o | | | | |
|---|---|---|---|---|---|
| | feeder | 0 ng/ml | 200 ng/ml | 400 ng/ml | 800 ng/ml |
| 0d | 0.95 | 0.95 | 0.95 | 0.95 | 0.95 |
| 7d | 48.8 | 45.2 | 56.1 | 50.1 | 60.9 |
| 14d | 163.9 | 208.5 | 229.5 | 262.6 | 404.1 |

### Example 2.2

### Effect of COMP-Ang1 in Maintaining of CD34+ and CD45+ Subpopulation

Expressions of CD34 and CD45 in CD34+ enriched cells were monitored during culture on dish or on feeder layer cells in different concentrations of COMP-Ang1. At day 7, subpopulation of CD34+ and CD45 cells was 73.5% in the dish culture condition, while it was 87.5% in the feeder layer culture condition. Addition of COMP-Ang1 increased percentage of subpopulation of CD34+ and CD45 cells in a dose-dependent manner in the feeder layer culture condition. From day 7 to day 14, subpopulation of CD34+ and CD45+ in total cells was deceased from 73.5% to 28.8% relatively in the dish culture, while it was decreased from 87.5% to 44.8% relatively in the feeder layer culture. Decreasing extents of subpopulation of CD34+ and CD45 cells from day 7 to day 14 were relatively similar between control buffer-treated group and different concentrations of COMP-Ang1-treated groups. However, it should be noted that subpopulation of CD34+ and CD45 cells was the highest in 800 ng/ml of COMP-Ang1-treated group.

The expanded stem cell population alone or in combination with other cell types or expanded umbilical cord blood cells, including total nuclear cells may be used to treat a variety of diseases. For instance, by way of example, and without limitation, the expanded cells infused into damaged heart muscle of a heart attack patient may generate new heart tissue and repair the damage. The stem cells may be infused into the hearts of patients with clogged arteries thus treating coronary artery disease. The stem cells may also aid in new blood vessel growth around blocked arteries, thus improving blood flow to the areas in the heart at risk of damage. Further, infusion of the expanded cells results in growth of new blood vessels around narrowed or damaged arteries in the limbs and restore impaired blood flow.

The expanded cells of the invention are also capable of treating nerve and brain damage. Human stem cells can mature into nerve cells thus treating a variety of neurological problems such as those brought on by strokes or traumatic injury or aging, such as multiple sclerosis, Parkinson's Disease and Alzheimer's and so forth.

All of the references cited herein are incorporated by reference in their entirety.

Those skilled in the art will recognize, or be able to ascertain using no more than routine experimentation, many equivalents to the specific embodiments of the invention specifically described herein. Such equivalents are intended to be encompassed in the scope of the claims.

What is claimed is:

1. A method for expanding a population of mammalian cells comprising contacting the cells with angiopoietin 1.

2. The method according to claim 1, wherein the angiopoietin 1 is a chimeric angiopoietin 1.

3. The method according to claim 2, wherein the angiopoietin is substituted at the N-terminus with a coiled-coil domain.

4. The method according to claim 3, wherein the coiled-coil domain is from cartilage oligomeric matrix protein (COMP).

5. The method according to claim 1, wherein the expansion is in vivo, in vitro or ex vivo.

6. The method according to claim 1, wherein the mammalian cell is a stem cell.

7. The method according to claim 6, wherein the stem cell is a totipotent or pluripotent stem cell.

8. The method according to claim 7, wherein the totipotent stem cell is embryonic stem cell.

9. The method according to claim 8, wherein the pluripotent stem cell is hematopoietic stem cell (HSC) or mesenchymal stem cell.

10. The method according to claim 9, wherein the HSC is without limitation CD34+ or CD45+.

11. The method according to claim 1, wherein the mammalian cell is obtained from umbilical cord blood.

12. The method according to claim 1, wherein the cell is the total nuclear cell of umbilical cord blood.

13. The method according to claim 1, wherein the mammal is human.

14. The method according to claim 1, wherein the angiopoietin 1 is delivered to the cell via genetic vector format wherein the polypeptide molecule is expressed in a host cell and is secreted into the population.

15. A container which contains (a) a compound of coiled coil domain linked to Ang 1; and (b) instructions that cells be expanded by contacting the cells with the compound.
